(19) United States
(12) Patent Application Publication
(10) Pub. No.: US 2006/0111724 A1
(43) Pub. Date: May 25, 2006

(54) Bone harvesting drill bit for oral surgery

(75) Inventor: Jean-Claude Yeung Wai Ping, Joinville Le Pont (FR)

Correspondence Address: YOUNG & THOMPSON, 745 SOUTH 23RD STREET, 2ND FLOOR, ARLINGTON, VA, 22202, US

(73) Assignee: YEUNG RESEARCH, JOINVILLE LE PONT (FR)

(21) Appl. No.: 11/168,316

(22) Filed: Jun. 29, 2005

(30) Foreign Application Priority Data
Nov. 24, 2004 (FR) 0412442
Mar. 15, 2005 (FR) 0502512

(51) Int. Cl. A61B 17/16

(52) U.S. Cl. 606/80

(57) ABSTRACT

A bone harvesting drill bit has a longitudinal axis and includes a proximal end portion adapted to be mounted for rotation in a chuck of a surgical handpiece. The drill bit has a distal end portion having a boring tip for boring into a bone harvesting site and an intermediate or lateral cutting zone located between the distal and proximal end portions and at least one substantially longitudinal cutting edge making an angle of less than 20° relative to the longitudinal axis, and at least one bone collecting zone adjoining the at least one longitudinal cutting edge and having a radial depth greater than the radius of the intermediate or lateral cutting zone.

### BACKGROUND OF THE INVENTION

1. Field of the Invention

The invention concerns harvesting or collecting morselized bone and in particular cortical and cancellous from bone harvesting sites in the oral cavity. Such bone is often used to reinforce recipient sites with inadequate bone height and width for implant placement.

2. Description of the Prior Art

Various devices have been developed for the harvesting of bone for use in oral surgery. Conventionally bone collecting trephines are employed. These comprise tubular cutters with a ring of teeth disposed along the distal edge. The proximal end portion of the cutter is mounted for rotational movement on a handpiece or counterangle for cutting a core from the desired bone harvesting site. The bone core or plug must be chopped or morselized before it can be transferred to a recipient site.

International patent application WO 02/41792 discloses a trephine of the foregoing type with a bone grinding blade located proximate to the distal end portion thereof for morselizing the bone as it enters the interior cavity defined by the trephine tube. The mandrill for the trephine has its own irrigation bore in order to avoid narcosis of the bone being cut.

U.S. Pat. No. 5,156,399 discloses a coring drill for harvesting bone from the iliac crest of the hip bone comprising a cylindrical hollow shaft and a half-conical tip or cutting head which morselizes the bone as it enters the hollow shaft. The coring drill bit operates at a relatively low speed of 200 rpm. The bit is then removed from the drill bit chuck and a plunger is introduced through the opening in the conical tip to remove the morselized bone through the proximal end of the hollow shaft.

U.S. Pat. No. 6,022,354 discloses a rotary drill for harvesting and morselizing bone fragments from a harvest site in the jaw or the iliac crest of the hip. This device includes a collection reservoir connected to a suction port for removing bone fragments and irrigation fluids from the harvest site.

U.S. Pat. No. 6,235,035 discloses a bone recovery surgical drill with an active portion including helicoidal-like grooves and a stop axially spaced from the active portion to limit penetration of drill and also defining with the active portion an annular bone recovery area for receiving bone residue carried by the grooves towards the recovery area.

In another known drill bit for producing bone fragments or particles, the cutting portion comprises the helicoidal cutting edges which define corresponding helicoidal grooves of limited depth and therefore of limited bone limited recovery capacity. Also, when the bone fragments reach the proximal end of the cutting portion, they may be lost before they are recovered.

FIG. 1 is a side elevation view of a further prior art drill bit (DB1) having a plurality of bone harvesting valleys at a distal end thereof and an irrigation bore (IB) and FIG. 2 is a cross-sectional view thereof. This known design has a substantially frustoconical (or cylindrical, not shown) intermediate or lateral part (IP) with a plurality of longitudinal (extending along generatrices of the frustoconical or cylindrical body) cutting edges (LCE) (typically three or four) circumferentially spaced from one another. The cutting edges define one outer edge of respective V-shaped troughs or valleys (VT) which are intended to collect morselized bone. These valleys are shallow in radial depth and the quantity of bone which may be harvested therein is correspondingly limited. The distal end has a pointed tip (PT) with an inclined cutting edge (ICE). Also, as the drill bit is designed to be operated at relatively high rotational speeds, it must be irrigated.

FIG. 3 is an elevation view showing another known drill bit (DB2) which has a V-shaped pointed tip (PT) at its distal end whose oblique leading edges define cutting edges (OCE) for boring into the bone. The pointed tip (PT) has opposed, parallel faces (F) and thus defines a blade. Each of the faces of the pointed tip is extended proximally by a narrow land (NL) to an abutment collar (AC) which limits penetration of the drill bit into the bone. Longitudinal edges bound each of the narrow lands (NL) and also extend proximally to the abutment collar. These longitudinal edges (LBE) are not honed blunt and have apparently no cutting function. As the drill bit is intended for high speed drilling it is equipped with a longitudinal irrigation bore (IB). Only very limited quantities of bone from the cutting edges of the pointed tip can collect on the narrow lands (NL) and/or faces of the pointed tip because of their small surface areas and because of the irrigation water which tends to flush off the bone therefrom.

### SUMMARY OF THE INVENTION

The object of the invention is to mitigate the drawbacks of known bone harvesting drill bits and provide an improved drill bit which is of relatively simple construction and manufacture with a satisfactory collecting capacity for morselized bone.

According to one aspect of the invention, there is provided a bone harvesting drill bit having a longitudinal axis and comprising a proximal end portion adapted to be mounted for rotation in a chuck of a surgical handpiece and a distal end portion having a boring tip for boring into a bone harvesting site. An intermediate or lateral cutting zone is located between the distal and proximal end portions and includes at least one longitudinal cutting edge making an angle of less than 20° relative to the longitudinal axis. At least one bone collecting zone adjoins the at least one longitudinal cutting edge and has a radial depth greater than the radius of the intermediate or lateral cutting zone of the drill bit.

According to an embodiment, the intermediate or lateral cutting zone is preferably substantially cylindrical and in any event has a distal taper less than 20° with respect to the longitudinal axis and preferably less than 10° or lower.

According to an embodiment, the intermediate or lateral cutting zone has two or three substantially parallel longitudinal cutting edges and respective adjoining valleys communicating with each other.

In this embodiment, there are one or two valleys, each valley having a valley wall terminating along one of said substantially opposed faces of the pointed tip.

According to an embodiment, the boring tip at the distal end portion is substantially V-shaped and comprises a substantially flat blade.

According to another aspect of the invention, there is provided a bone harvesting drill bit having a longitudinal axis and comprising a proximal end portion adapted to be mounted for rotation in a chuck of a surgical handpiece and a distal end portion having a boring tip and at least one cutting edge for boring into a bone harvesting site. An intermediate or lateral cutting zone is located between the distal and proximal end portions and including at least two substantially longitudinal cutting edges making an angle of less than 20° relative to the longitudinal axis. A bone collecting zone adjoins each of the longitudinal cutting edges and defines a central cavity inside the intermediate or lateral cutting zone.

According to an embodiment, the cutting edges are defined by leading edges of arcuate arms, all extending in the same circumferential direction. The respective arcuate arms are preferably joined together proximate to the distal end portion and proximate to the proximal end portion of the drill bit.

These and other features will become apparent from the following description given by way of example with reference to FIGS. 4-13 of the accompanying drawings.

### BRIEF DESCRIPTION OF THE DRAWINGS

FIG. 4 is a side elevation of a bone harvesting drill bit embodying a first aspect of the present invention;

FIG. 5 is a cross sectional view of bone harvesting drill bit of FIG. 4 taken along line V-V in FIG. 4;

FIG. 6 is a distal end view of the bone harvesting drill bit of FIG. 4;

FIG. 7 is a side elevation of another embodiment of the bone harvesting drill bit according to the invention having two longitudinal cutting edges and adjoining bone harvesting valleys;

FIG. 8 is a cross sectional view of the drill bit of FIG. 7 through the intermediate or lateral cutting zone of the bone harvesting drill bit along line VIII-VIII in FIG. 7;

FIG. 9 is a distal end view of the bone harvesting drill bit of FIG. 7;

FIG. 10 is a side elevation of another embodiment of the bone harvesting drill bit according to the invention, having three longitudinal cutting edges and adjoining bone harvesting valleys;

FIG. 11 is a cross sectional view of the drill bit of FIG. 10 through the intermediate or lateral cutting zone of the bone harvesting drill bit along line XI-XI of FIG. 10;

FIG. 12 is a distal end portion view of the bone harvesting drill bit of FIG. 10; and

FIG. 13 is a detailed view of a variant of the pointed tip having an arcuate cutting edge.

### DETAILED DESCRIPTION OF PREFERRED EMBODIMENTS

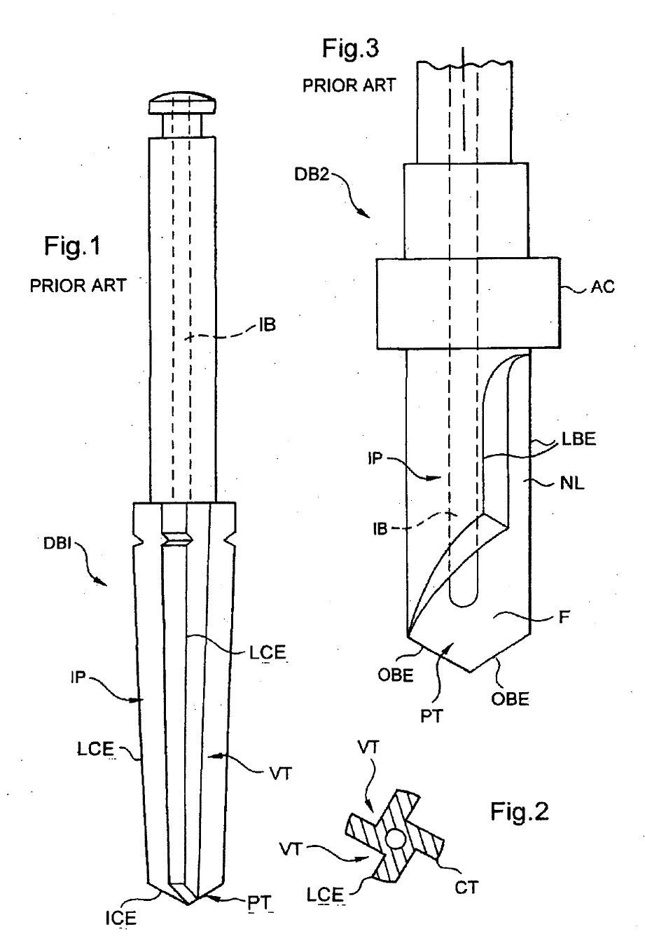

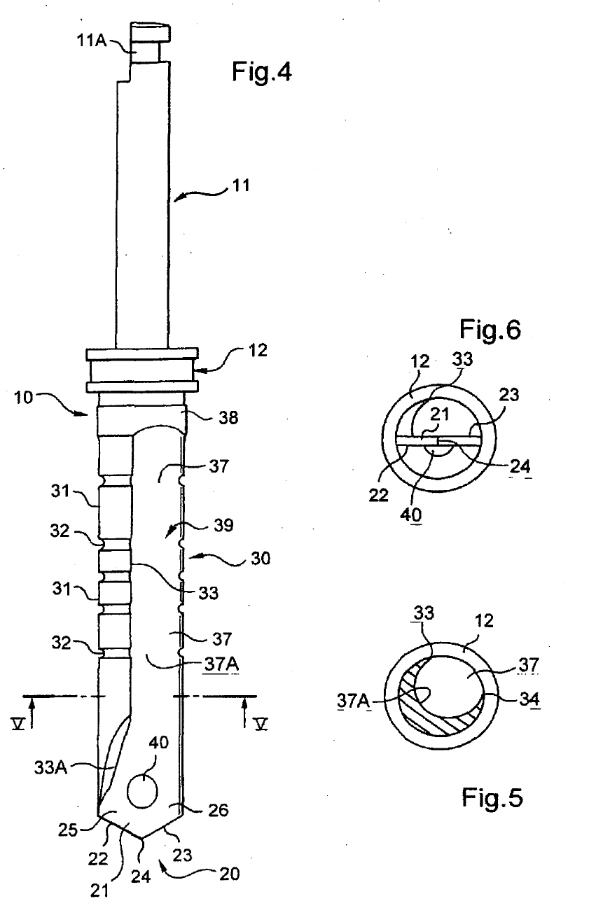

A first embodiment of the bone harvesting drill bit according to the present invention is shown in FIGS. 4-6 and designated by reference 10. Drill bit 10 is made of stainless steel or other suitable metal or alloys and is generally of cylindrical in configuration and has a substantially cylindrical proximal shank portion 11, for example, with a conventional coupling groove 11A surmounted by a first annular collar at its free upper end for mounting in the chuck of a conventional counterangle or surgical handpiece (not shown) in particular for dental surgery. The drill bit 10 also comprises a distal end portion 20 for boring into the harvesting site and a generally cylindrical intermediate or lateral cutting zone 30 located axially between the distal end portion 20 and proximal shank 11 portion of the drill bit.

As is conventional and known per se, a second annular collar or abutment 12 of enlarged diameter is located at the lower end of the proximal shank portion 11, remote from the coupling groove 11A, adapted to limit the depth of penetration of the drill bit into a bone harvesting site. The second annular collar may carry identification information relating, for example, to the diameter of the particular bone harvesting drill bit.

The distal end portion 20 generally comprises a boring tip including, as illustrated, a flat or platelike blade with a transverse thickness from about 0.35 mm to about 0.60 mm and preferably about 0.45 mm. The distal end portion 20 has a pointed boring tip 21 including distally converging, obliquely honed cutting edges 22, 23. The cutting edges 22, 23 meet at the distal extremity defined by a distal transverse ridge 24 located on the longitudinal axis of the drill bit. The angle formed by the cutting edges (as shown in FIG. 4) is for example between about 90° and about 140°. The obliqueness of the obliquely honed cutting edges are angled in the same direction so each of them produces the same cutting action when the drill bit rotates in the given direction of rotation (i.e. clockwise or counterclockwise). The distal end portion is designed for boring into cortical and cancellous bone in the oral cavity.

In the illustrated embodiment, the intermediate or lateral cutting zone 30 of the drill bit 10 has a generally cylindrical outer surface which, as shown, includes a plurality of cylindrical segments 31 of the same or different axial lengths spaced from each other by annular grooves 32. Alternatively, the outer surface of the intermediate or lateral cutting zone may taper distally. The angle of such a taper or frustoconical surface is less than 20° relative to the longitudinal axis and preferably less than 10° and in practice 50 or less. The annular grooves 32 between the cylindrical segments provide a visual indication of the depth of the penetration of the pointed tip into the bone harvesting site. For example, the annular grooves can be located at, for example, 8, 10, 11.5, 13 and 15 mm from the distal extremity or ridge 43. The alternating cylindrical segments and grooves are optional. In lieu thereof, the outer surface of the intermediate or lateral cutting zone may be at continuous cylindrical or frustoconical shape and devoid of such annular grooves. In this case, the longitudinal cutting edge 33 is devoid of notches corresponding to the annular groove.

The intermediate or lateral cutting zone 30 comprises at least one longitudinal cutting edge 33 and a single longitudinal cutting edge in the case of the embodiment of FIG. 4. The generally longitudinal cutting edge(s) is obliquely honed (not shown) in the direction of intended rotation, here counterclockwise. The longitudinal cutting edge 33 terminates at its distal end at an oblique wall 33A which merges into the corresponding face of the pointed tip. A valley or trough 37 adjoins each longitudinal cutting edge 33. As illustrated in FIG. 4, there is a single valley or trough 37 and the valley walls define a generally cylindrical concave surface eccentric in relation to the cylindrical outer surface of the intermediate or lateral cutting zone. The valley or trough 37 extends from the longitudinal cutting edge 33 to an opposite generally longitudinal edge 39. This opposite longitudinal edge is not a cutting edge in the illustrated embodiment. Alternatively, if longitudinal edge 39 is honed as a cutting edge, the drill bit may be operated in either direction of rotation. The part circumferential opening of the valley over the outer cylindrical/frustoconical wall of the intermediate or lateral cutting zone between the longitudinal edges 34 and 39 is generally at least 120°. The depth of the valley or trough is greater than the radius of the outer surface of the intermediate or lateral cutting zone in order to define a relatively deep cavity with a sufficient morselized bone recovery capacity. As illustrated, the bone recovery capacity of the cavity is greater than 50% of the volume defined by a theoretical cylinder or frustocone with a continuous sidewall having the same radial dimensions as the intermediate or lateral cutting zone.

An opening 40 is defined at the intersection of the distal portion with the intermediate or lateral cutting zone owing to the depth of the trough or valley and the fact that the distal end portion is a thin blade and located at the longitudinal axis of the drill bit. The valley or trough 37 extends from the distal end portion 20 to an abutment 38 which, as illustrated, is immediately below the annular collar 22. The capacity of the cavity will depend on the diameter of the drill bit. Typically, the drill bit diameter will be between 2.5 and 4.5 mm. In practice, a plurality of different diameter bone harvesting drill bits to be used successively or separately are grouped together as a kit.

While the generally cylindrical concave valley wall surface 37A is shown which may cut with a milling machine, other valley wall configurations may be adopted, for example of V-shaped or U-shaped cross section cut with a lathe. Likewise, the proximal end wall of trough or valley 37 may be of any form.

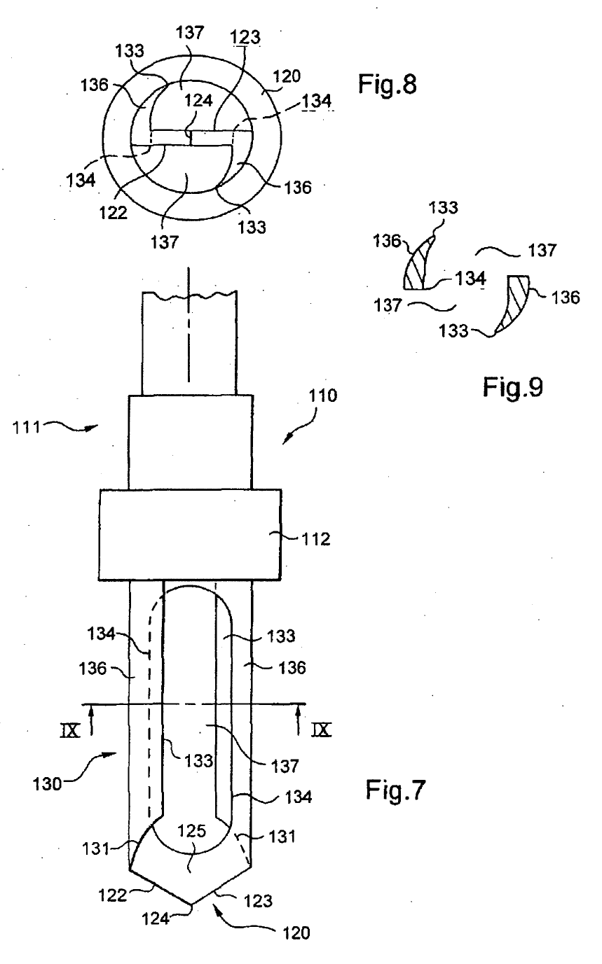

A second embodiment of the bone harvesting drill bit is shown in FIGS. 7-9 and designated by reference 110. Drill bit 110 is also made of stainless steel or other suitable metal or alloys and is of generally cylindrical configuration comprises a cylindrical proximal end portion 111 (a part only thereof being illustrated) adapted to be mounted in the chuck of a conventional counterangle or handpiece (not shown). The drill bit further comprises a generally flat or platelike distal end portion 120 and a generally cylindrical intermediate or lateral cutting zone 130 located between the distal end portion and proximal end portion 111 of the drill bit.

As is conventional and known per se, and noted above, the proximal end portion comprises inter alia an annular collar 112 of enlarged diameter which in the present drill bit is adapted to limit the penetration of the drill bit into a harvesting site.

The distal end portion 120 is generally flat or platelike with a transverse thickness as indicated above. The distal end portion has a pointed boring tip 121 having distally converging cutting edges 122, 123. The cutting edges are obliquely honed from opposite sides of the generally flat or platelike distal end portion on leading edges thereof with respect to the desired direction of rotation of the drill bit, here assumed counterclockwise. The obliquely honed cutting edges 122, 123 meet along transverse ridge 124. The respective flat faces 125 of the distal end portion are delimited by an inclined or arcuate (as shown) endwall 131 of the intermediate or lateral cutting zone 130.

In this illustrated embodiment, the intermediate or lateral cutting portion 130 of the drill bit 110 comprises a pair of diametrically opposed longitudinal cutting edges 133. Each of these cutting edges is obliquely honed in the direction of intended rotation, here assumed counterclockwise, and runs from the respective converging cutting edges 122, 123 in the proximal direction along a generatrices of the intermediate or lateral cutting zone. The intermediate or lateral cutting zone 130 in this embodiment comprises two part cylindrical sections. Each of these cylindrical sections comprises a second longitudinal edge 134 generally parallel to the longitudinal cutting edge. This second longitudinal edge 134 is not a cutting edge in this embodiment and thus only the drill bit will perform lateral cutting as it turns in one direction of rotation, here counterclockwise. Alternatively, the second longitudinal edge 134 of each one or both part cylindrical sections may also be a cutting edge in which case the drill bit will perform lateral cutting as it turns in either direction of rotation.

Alternatively, the part cylindrical sections may be slightly frustoconical with an angle limited as described above in connection with the first embodiment. A valley or trough 137 adjoins each of the longitudinal cutting edges 133 and is defined between substantially longitudinally and radially inwardly valley walls extending from the respective longitudinal cutting edges 133 and an opposite substantially longitudinally and radially inwardly valley wall extending from the adjoining second longitudinal edge of the second cylindrical section. As illustrated, each of the valleys or troughs defined between a pair of longitudinally and radially inwardly extending valley walls on the cylindrical sections opens into a common interior cavity extending from the collar 112 to the distal end portion 120. Alternatively, and not shown, each of the troughs or valleys may be closed at the junction of its valley walls and out of communication with the other trough or valley.

The valleys or troughs preferably extend over at least 90° and more preferably at least over 120° corresponding to the circumferential angle between the associated longitudinal cutting edge and opposite longitudinal edges, thereby providing a large internal volume for collecting morselized bone. Where the valleys or troughs are in communication with each other across the longitudinal axis, the total bone collecting volume of the drill bit may be well in excess of the half volume enclosed by a theoretical cylinder or frustocone having the diameter of intermediate or lateral cutting zone.

A third preferred embodiment of the bone harvesting drill bit is shown in FIGS. 7-9 and designated by reference 210. Drill bit 210 is also made of stainless steel or other suitable metal or alloys and is of generally cylindrical configuration comprises a cylindrical proximal end portion 211 (a portion only thereof being illustrated) adapted to be mounted in the chuck of a conventional counterangle or handpiece (not shown). The drill bit further comprises a generally flat distal end portion 220 and a generally cylindrical intermediate or lateral cutting zone 230 located between the distal end portion and proximal end portion 211 of the drill bit.

As conventional and known per se and noted above, such a proximal end portion comprises, inter alia, an annular collar 212 of enlarged diameter which in the present drill bit is adapted to limit the introduction of thereof into a harvesting site.

The distal end portion 220 is generally flat or platelike with a transverse thickness of about 0.4 to 1.5 mm. The distal end portion has a pointed boring tip 221 having distally converging cutting edges 222, 223. The cutting edges are obliquely honed from opposite sides of the generally flat or platelike distal end portion on leading edges thereof with respect to the desired direction of rotation of the drill bit, here assumed counterclockwise. The obliquely honed cutting edges 222, 223 meet along a three-armed transverse ridge 224. The distal end portion is designed for boring into cortical and cancellous bone in the oral cavity. Each of the respective flat faces 225 of the distal end portion is delimited by an inclined or arcuate (as shown) endwall 231 of the intermediate or lateral cutting zone 230.

As illustrated in the variant of FIG. 13, instead of the rectilinear obliquely honed cutting edges of the boring tip in the preceding embodiments, the pointed boring tip 320 comprises one or more convex curved obliquely honed cutting edges at the distal end of the drill bit below the intermediate or lateral cutting zone 330.

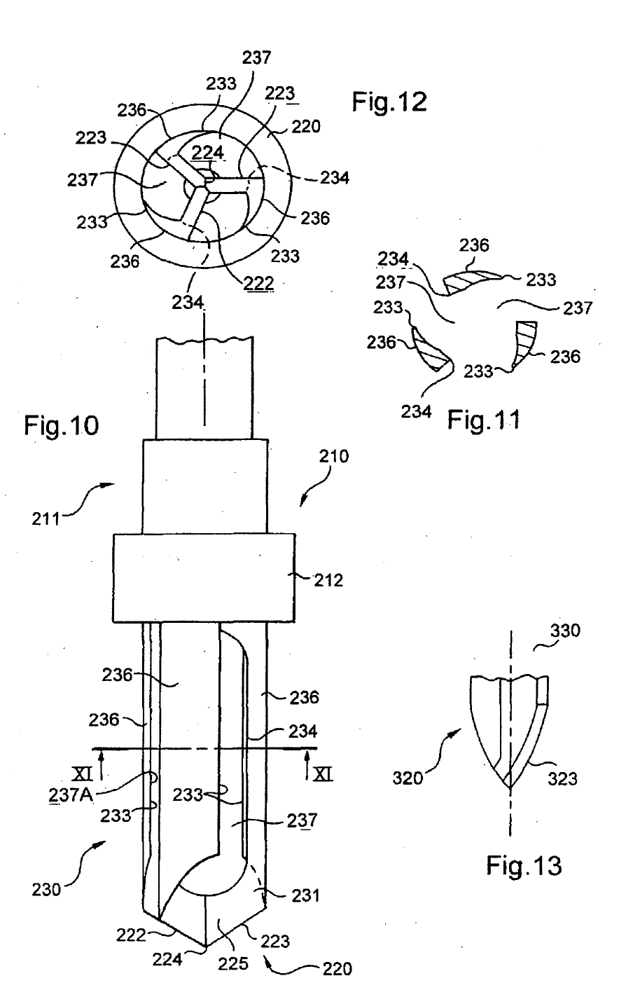

Returning to the third illustrated embodiment of FIGS. 10-12, the intermediate or lateral cutting portion 230 of the drill bit 210 comprises three circumferentially spaced longitudinal cutting edges 233. Each of these cutting edges is obliquely honed in the direction of intended rotation, here assumed counterclockwise, and runs from the respective converging distal cutting edges 222, 223 in the proximal direction along a generatrix of the intermediate or lateral cutting zone. The intermediate or lateral cutting zone 230 comprises three part cylindrical sectors (236) extending circumferentially between a longitudinal cutting edge and an opposite (non cutting) longitudinal edge. Each of these cylindrical sectors comprises a longitudinal cutting edge 233 generally parallel to each other. A second longitudinal edge 234 of each valley is not a cutting edge, as illustrated, and is formed on the adjacent sector. In this case the drill bit will perform lateral cutting only as it turns in a direction of rotation, here counterclockwise. Alternatively, the second opposite longitudinal edges may also be honed into cutting edges in which case the drill bit will perform lateral cutting as it turns in either direction.

Alternatively, the part cylindrical sectors (235) may be frustoconical with an angle not greater than 20° with respect to the longitudinal axis of the drill bit and preferably not greater than 10° and most preferably 5° or less, with respect to the longitudinal axis of the drill bit.

A valley or trough 237 adjoins each of the three longitudinal cutting edges 233 and is defined between substantially longitudinally and radially inwardly extending valley walls from respective pairs of longitudinal edges 233, 234. As illustrated, each of the valleys or troughs defined between a pair of longitudinally and radially inwardly extending valley walls on the adjacent cylindrical sectors opens into a common interior cavity extending from the collar 212 to the distal end portion 220.

The valleys or troughs preferably extend over at least 60° and more preferably at least over 90° thereby providing a large internal cavity with a volume for collecting sufficient amounts of morselized bone. Where the valleys or troughs are in communication with each other across the longitudinal axis, the total bone collecting volume of the drill bit is in excess of half the volume enclosed by a theoretical cylinder or frustocone having the diameter of intermediate or lateral cutting zone.

In the illustrated embodiments, no irrigation is required as the drill bit is rotated at a speed less than about 200 rpm and preferably less than about 150 rpm and most preferably between 60-120 rpm.

For rotational speeds, in excess of the preferred values, an irrigation bore (not shown) may be machined along the longitudinal axis of the drill bit from its extreme proximal end to a transverse opening extending through the platelike distal end portion into which the irrigation bore opens.

After harvesting from the harvesting site, the morselized bone may be transferred to a receptor site or the implant sites by means of the usual known instruments.

It will be appreciated that the scope of the present invention is not limited to the details or numerical values of the embodiments given above by way of example but to the contrary extends to modifications that will be apparent to one having ordinary skill in the art.

There is claimed:

1. A bone harvesting drill bit having a longitudinal axis and comprising a proximal end portion adapted to be mounted for rotation in a chuck of a surgical handpiece and a distal end portion having a boring tip for boring into a bone harvesting site, an intermediate or lateral cutting zone located between the distal and proximal end portions and including at least one substantially straight longitudinal cutting edge making an angle of less than 20° relative to the longitudinal axis, and at least one bone collecting zone adjoining the at least one longitudinal cutting edge and having a radial depth greater than the radius of the intermediate or lateral cutting zone.

2. A bone harvesting drill bit according to claim 1, wherein the pointed tip at the distal end portion is substantially V-shaped and comprises a substantially flat blade.

3. A bone harvesting drill bit according to claim 2, wherein the blade has a thickness from about 0.35 mm to about 0.60 mm.

4. A bone harvesting drill bit according to claim 3, wherein the blade has a thickness of about 0.45 mm.

5. A bone harvesting drill bit according to claim 2, wherein at least one edge of the pointed tip comprises a cutting edge.

6. A bone harvesting drill bit according to claim 5, wherein both edges of the pointed tip are cutting edges and at an angle to each other of between about 90° and about 140°.

7. A bone harvesting drill bit according to claim 1, wherein the distal end portion has a pair of substantially opposed faces, said bone collecting zone defining a valley having a valley wall terminating along one of said substantially opposed faces.

8. A bone harvesting drill bit according to claim 7, wherein there are two said valleys, each said valley having a valley wall terminating along one of said substantially opposed faces.

9. A bone harvesting drill bit according to claim 7, wherein said valley has a generally U-shape cross section.

10. A bone harvesting drill bit according to claim 7, wherein said valley has a generally V-shape cross section.

11. A bone harvesting drill bit according to claim 1, wherein the intermediate or lateral cutting zone has a substantially cylindrical external wall.

12. A bone harvesting drill bit according to claim 1, wherein the intermediate or lateral cutting zone has a distal taper less than 10° with respect to the longitudinal axis.

13. A bone harvesting drill bit according to claim 1, wherein the intermediate or lateral cutting zone has a distal taper less than 5° with respect to the longitudinal axis.

14. A bone harvesting drill bit according to claim 1, wherein the intermediate or lateral cutting zone has two circumferentially spaced substantially parallel cutting edges and respective adjoining harvesting valleys communicating with each other.

15. A bone harvesting drill bit according to claim 1, wherein the at least one bone cutting zone has an internal volume in excess of the volume of a cylinder or frustocone having the diameter equal to that of the intermediate or lateral cutting zone.

16. A bone harvesting drill bit having a longitudinal axis and comprising a proximal end portion adapted to be mounted for rotation in a chuck of a surgical handpiece and a distal end portion, having a boring tip and at least one cutting edge for boring into a bone harvesting site, an intermediate or lateral cutting zone located between the distal and proximal end portions and including at least two substantially longitudinal cutting edges making an angle of less than 20° relative to the longitudinal axis, and a bone collecting zone adjoining the longitudinal cutting edges and defining a central cavity inside the intermediate or lateral cutting zone.

17. A bone harvesting drill bit according to claim 16, wherein the cutting edges are defined by leading edges of arcuate arms, extending in the same circumferential direction.

18. A bone harvesting drill bit according to claim 17, wherein the respective arcuate arms are joined together proximate to the distal end portion and proximate to the proximal end portion of the drill bit.

19. A bone harvesting drill bit according to claim 18, wherein the arcuate arms are joined together by respective radial arms.

20. A bone harvesting drill bit according to claim 16, wherein the pointed tip at the distal end portion is substantially V-shaped and comprises a substantially flat blade.

21. A bone harvesting drill bit according to claim 16, wherein the blade has a thickness from about 0.35 mm to about 0.60 mm.

22. A bone harvesting drill bit according to claim 16, wherein the pointed tip has two cutting edges at an angle between about 90° and about 140°.

23. A bone harvesting drill bit according to claim 16, wherein the distal end portion has a pair of substantially opposed faces.

24. A bone harvesting drill bit according to claim 16, wherein the intermediate or lateral cutting zone has a substantially cylindrical external wall.

25. A bone harvesting drill bit according to claim 16, wherein the intermediate or lateral cutting zone has a distal taper less than 10° with respect to the longitudinal axis.

26. A bone harvesting drill bit according to claim 16 wherein the intermediate zone has a distal taper less than 5° with respect to the longitudinal axis.

27. A bone harvesting drill bit according to claim 16, wherein the at least one bone cutting zone has an internal volume in excess of the volume of a cylinder or frustocone having the diameter equal to that of the intermediate or lateral cutting zone.
